(19) United States
(12) Patent Application Publication
(10) Pub. No.: US 2026/0036706 A1
(43) Pub. Date: Feb. 5, 2026

(54) IMAGE ACQUISITION DEVICE AND IMAGE ACQUISITION METHOD

(71) Applicant: HAMAMATSU PHOTONICS K.K., Hamamatsu-shi, Shizuoka (JP)

(72) Inventors: Ryosuke OTA, Hamamatsu-shi, Shizuoka (JP); Kibo OTE, Hamamatsu-shi, Shizuoka (JP); Fumio HASHIMOTO, Hamamatsu-shi, Shizuoka (JP); Tomoo INUBUSHI, Hamamatsu-shi, Shizuoka (JP); Yuya ONISHI, Hamamatsu-shi, Shizuoka (JP); Takashi ISOBE, Hamamatsu-shi, Shizuoka (JP)

(73) Assignee: HAMAMATSU PHOTONICS K.K., Hamamatsu-shi, Shizuoka (JP)

(21) Appl. No.: 18/998,316

(22) PCT Filed: Aug. 28, 2023
(86) PCT No.: PCT/JP2023/030969

§ 371 Date: Jan. 24, 2025

(30) Foreign Application Priority Data
Aug. 30, 2022 (JP) 2022-136575

(51) Int. Cl. G01T 1/29; A61B 6/03

(52) U.S. Cl. CPC G01T 1/2985; A61B 6/037

(57) ABSTRACT

An image acquisition apparatus includes a measurement unit and a processing unit. The processing unit, for each coincidence event in which a first detector and a second detector perform coincidence detection of a pair of gamma-ray photons generated by an electron positron annihilation event in a positron emitting radionuclide, assuming that the gamma-ray photon arriving at one detector is a gamma-ray photon arriving without being Compton scattered in a subject, and the gamma-ray photon arriving at another detector is a gamma-ray photon arriving after being Compton scattered in the subject, determines a position at which the gamma-ray photon is Compton scattered in the subject based on a detection position and a detection time of the gamma-ray photon by each of the first detector and the second detector and a position of the positron emitting radionuclide.

### TECHNICAL FIELD

The present disclosure relates to an image acquisition apparatus and an image acquisition method.

### BACKGROUND ART

A nuclear medicine diagnostic apparatus such as a positron emission tomography (PET) apparatus and a single photon emission computed tomography (SPECT) apparatus can acquire a tomographic image of a subject into which a drug which is labeled with a positron emitting radionuclide or a single photon emitting radionuclide is injected. The tomographic image acquired by each of the nuclear medicine diagnostic apparatuses described above represents a distribution of positron emitting radionuclides or single photon emitting radionuclides (a distribution of the drug) in the subject, and can be used for performing diagnosis of a health condition of the subject and the like.

An X-ray CT apparatus can also acquire a three-dimensional tomographic image of the subject. The tomographic image acquired by the X-ray CT apparatus represents anatomical information of the subject. Hereinafter, it is assumed that the tomographic image is a three-dimensional tomographic image.

In order to improve an image quality of a PET image, it is carried out that the PET apparatus and the X-ray CT apparatus are used in combination, and the PET image is corrected by using the anatomical information acquired by the X-ray CT apparatus. However, the X-ray CT apparatus is expensive, and thus, research and development of an inexpensive apparatus capable of acquiring the tomographic image representing the anatomical information of the subject are being conducted.

Each of Non Patent Document 1 and Patent Document 1 describes an apparatus capable of acquiring both the tomographic image representing the distribution of the positron emitting radionuclides in the subject and the tomographic image representing the anatomical information.

The apparatus described in Non Patent Document 1 has a configuration of the PET apparatus in which a large number of detectors are arranged around a measurement space in which the subject is placed. In the above apparatus, a detector having an LSO (Lu₂SiO₅: Ce) scintillator is used, and a gamma-ray transmitted through the subject in gamma-rays of an energy of 307 keV or 202 keV emitted from 176Lu contained in the LSO scintillator of each detector is detected by another detector. Further, the above apparatus acquires the tomographic image representing the anatomical information of the subject by performing image reconstruction processing based on the detection result of the gamma-rays of the energy of 307 keV or 202 keV.

The apparatus described in Patent Document 1 uses an electron tracking Compton camera (ETCC). In a Compton camera, in general, it is estimated that a gamma-ray arrives from any position on a conical surface called a Compton cone based on energy information in each of a scatterer and an absorber. On the other hand, in the ETCC, it is possible to uniquely identify a gamma-ray arrival direction by tracking a track of a recoil electron by using a gas detector.

The above apparatus detects, by the ETCC, the arrival direction of the gamma-ray having an energy which is reduced by Compton scattering in the subject in gamma-rays generated in the subject into which the drug labeled with the positron emitting radionuclide is injected. Further, in the above apparatus, the tomographic image representing the anatomical information of the subject is acquired by using an analytical method or a statistical method based on the detection result of the gamma-ray arrival direction by the ETCC, that is, by performing the image reconstruction processing.

### CITATION LIST

### Patent Literature

Patent Document 1: Japanese Patent Publication No. 6990412

### Non Patent Literature

Non Patent Document 1: Mohammadreza Teimoorisichani et al., “A CT-less approach to quantitative PET imaging using the LSO intrinsic radiation for long-axial FOV PET scanners”, Med. Phys. Vol. 49, pp. 309-323, 2022
Non Patent Document 2: Gerard Arino-Estrada et al., “First Cerenkov charge-induction (CCI) TIBr detector for TOF-PET and proton range verification”, Phys. Med. Biol. 64 175001, 2019

### SUMMARY OF INVENTION

### Technical Problem

In both the techniques described in Non Patent Document 1 and Patent Document 1, it is necessary to perform the image reconstruction processing based on the gamma-ray detection result in order to acquire the tomographic image representing the anatomical information of the subject. In the tomographic image acquired by the image reconstruction processing, the image quality is reduced due to the image reconstruction processing, and the anatomical information is degraded.

An object of the present invention is to provide an image acquisition apparatus and an image acquisition method capable of acquiring a tomographic image representing anatomical information of a subject without performing image reconstruction processing.

### Solution to Problem

An embodiment of the present invention is an image acquisition apparatus. The image acquisition apparatus includes (1) a measurement unit including a first detector and a second detector each for detecting a gamma-ray photon, and for outputting a signal indicating a detection position and a detection time when each of the first detector and the second detector detects the gamma-ray photon; and (2) a processing unit for processing the signal output from each of the first detector and the second detector, and (3) the measurement unit, in a first measurement mode, in a state in which a subject is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing unit, in the first measurement mode, for each coincidence event in which the first detector and the second detector perform coincidence detection of a pair of gamma-ray photons generated by an electron positron annihilation event in the positron emitting radionuclide, assuming that the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide, and creates a first tomographic image representing a distribution of Compton scattering positions in the subject respectively determined for a plurality of coincidence events.

An embodiment of the present invention is an image acquisition apparatus. The image acquisition apparatus includes (1) a measurement unit including a first detector and a second detector each for detecting a gamma-ray photon, and for outputting a signal indicating a detection position and a detection time when each of the first detector and the second detector detects the gamma-ray photon; and (2) a processing unit for processing the signal output from each of the first detector and the second detector, and (3) the measurement unit, in a state in which a subject into which a drug labeled with a positron emitting radionuclide is injected is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing unit, for each coincidence event in which the first detector and the second detector perform coincidence detection of a pair of gamma-ray photons generated by an electron positron annihilation event in the positron emitting radionuclide, (a) in a case in which the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide placed between the first detector or the second detector and the subject, (b) in a case in which the gamma-ray photons arriving at both the first detector and the second detector are gamma-ray photons arriving without being Compton scattered in the subject, determines a position at which the annihilation event occurs based on the detection position and the detection time of the gamma-ray photon by each of the first detector and the second detector, (c) creates a first tomographic image representing a distribution of Compton scattering positions in the subject respectively determined for a plurality of coincidence events, creates a second tomographic image representing a distribution of annihilation event occurrence positions in the subject respectively determined for a plurality of coincidence events, and corrects the second tomographic image based on the first tomographic image.

An embodiment of the present invention is an image acquisition method. The image acquisition method includes (1) a measurement step of using a first detector and a second detector each for detecting a gamma-ray photon, and outputting a signal indicating a detection position and a detection time when each of the first detector and the second detector detects the gamma-ray photon; and (2) a processing step of processing the signal output from each of the first detector and the second detector, and (3) the measurement step, in a first measurement mode, in a state in which a subject is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing step, in the first measurement mode, for each coincidence event in which the first detector and the second detector perform coincidence detection of a pair of gamma-ray photons generated by an electron positron annihilation event in the positron emitting radionuclide, assuming that the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide, and creates a first tomographic image representing a distribution of Compton scattering positions in the subject respectively determined for a plurality of coincidence events.

An embodiment of the present invention is an image acquisition method. The image acquisition method includes (1) a measurement step of using a first detector and a second detector each for detecting a gamma-ray photon, and outputting a signal indicating a detection position and a detection time when each of the first detector and the second detector detects the gamma-ray photon; and (2) a processing step of processing the signal output from each of the first detector and the second detector, and (3) the measurement step, in a state in which a subject into which a drug labeled with a positron emitting radionuclide is injected is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing step, for each coincidence event in which the first detector and the second detector perform coincidence detection of a pair of gamma-ray photons generated by an electron positron annihilation event in the positron emitting radionuclide, (a) in a case in which the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide placed between the first detector or the second detector and the subject, (b) in a case in which the gamma-ray photons arriving at both the first detector and the second detector are gamma-ray photons arriving without being Compton scattered in the subject, determines a position at which the annihilation event occurs based on the detection position and the detection time of the gamma-ray photon by each of the first detector and the second detector, (c) creates a first tomographic image representing a distribution of Compton scattering positions in the subject respectively determined for a plurality of coincidence events, creates a second tomographic image representing a distribution of annihilation event occurrence positions in the subject respectively determined for a plurality of coincidence events, and corrects the second tomographic image based on the first tomographic image.

### Advantageous Effects of Invention

According to the embodiments of the present invention, it is possible to acquire a tomographic image representing anatomical information of a subject without performing image reconstruction processing.

### BRIEF DESCRIPTION OF DRAWINGS

FIG. 1 is a diagram illustrating a configuration of an image acquisition apparatus 1A according to a first embodiment (in particular, a diagram for describing acquisition of a first tomographic image in a first measurement mode).

FIG. 2 is a diagram illustrating a method of determining a position at which a gamma-ray photon is Compton scattered in a subject.

FIG. 3 is a diagram illustrating the configuration of the image acquisition apparatus 1A according to the first embodiment (in particular, a diagram for describing acquisition of a second tomographic image in a second measurement mode).

FIG. 4 is a diagram illustrating a configuration of an image acquisition apparatus 1B according to a second embodiment.

FIG. 5 is a diagram illustrating a configuration of an image acquisition apparatus 1C according to a third embodiment.

FIG. 6 is a diagram illustrating a configuration of an image acquisition apparatus 1D according to a fourth embodiment.

FIG. 7 is a diagram illustrating a configuration of an image acquisition apparatus 1E according to a fifth embodiment.

FIG. 8 is a diagram illustrating a configuration of an image acquisition apparatus 1F according to a sixth embodiment.

FIG. 9 includes (a), (b) diagrams for describing a change of a field of view of an apparatus in the case in which a positron emitting radionuclide 81 is moved in a direction parallel to a detection surface of a first detector 11 in a measurement unit 10F of the image acquisition apparatus 1F according to the sixth embodiment.

FIG. 10 includes (a), (b) diagrams for describing an image quality of a first tomographic image in the case in which the positron emitting radionuclide 81 is moved in a direction perpendicular to the detection surface of the first detector 11 in the measurement unit 10F of the image acquisition apparatus 1F according to the sixth embodiment.

FIG. 11 is a diagram illustrating a configuration and an arrangement of a measurement unit assumed in a simulation.

FIG. 12 is a diagram illustrating a configuration of a phantom assumed as a subject 90 in the simulation.

FIG. 13 is a diagram showing a first tomographic image acquired in the simulation.

### DESCRIPTION OF EMBODIMENTS

Hereinafter, embodiments of an image acquisition apparatus and an image acquisition method will be described in detail with reference to the accompanying drawings. In the description of the drawings, the same elements will be denoted by the same reference signs, and redundant description will be omitted. The present invention is not limited to these examples, and the Claims, their equivalents, and all the changes within the scope are intended as would fall within the scope of the present invention.

### First Embodiment

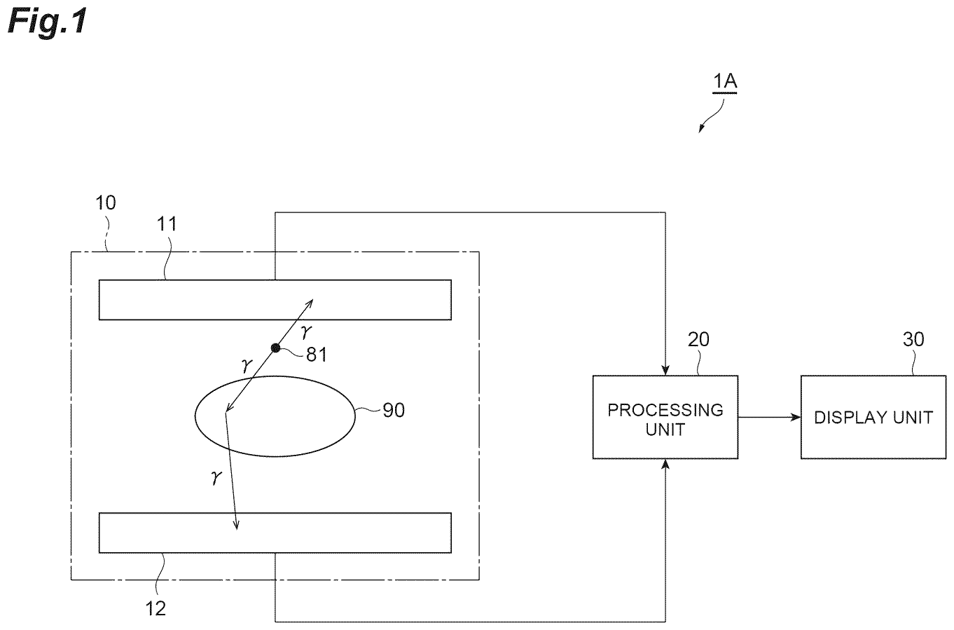

FIG. 1 is a diagram illustrating a configuration of an image acquisition apparatus 1A according to a first embodiment. The image acquisition apparatus 1A includes a measurement unit 10, a processing unit 20, and a display unit 30. The measurement unit 10 includes a first detector 11 and a second detector 12 disposed opposite to each other with a subject 90 interposed therebetween. Each of the first detector 11 and the second detector 12 is a detector for detecting a gamma-ray photon, and outputs a signal indicating a detection position and a detection time when the gamma-ray photon is detected.

The processing unit 20 processes the signal output from each of the first detector 11 and the second detector 12, and creates a tomographic image of the subject 90. The display unit 30 displays the tomographic image or the like created by the processing unit 20. The processing unit 20 and the display unit 30 may be configured by, for example, a computer.

As each of the first detector 11 and the second detector 12, for example, a Cherenkov detector is used. The Cherenkov detector includes a Cherenkov radiator (for example, lead glass, lead fluoride PbF₂, hafnium oxide HfO₂, or the like) and a microchannel plate photomultiplier tube (MCP-PMT). The Cherenkov detector may be configured by, for example, a two-dimensional array of small detectors each having no position detection capability, or may have a configuration in which the Cherenkov radiator and a multi-anode MCP-PMT are used in combination.

Further, the Cherenkov detector may have a configuration of using a slow scintillator such as BGO (Bi₄Ge₃O₁₂) as the Cherenkov radiator. The slow scintillator interacts with the gamma-ray to first emit Cherenkov light, and then emit scintillation light, and thus, it can be used as the Cherenkov radiator to achieve the high temporal resolution. In this case, an inexpensive detector can be configured as compared with the case of using the LSO scintillator or the like.

Further, as each of the first detector 11 and the second detector 12, for example, a semiconductor detector with the high temporal resolution may be used. The semiconductor detector having the high temporal resolution is, for example, a detector using thallium bromide (TlBr), and includes an electrode for charge collection and a high temporal resolution photodetector. The above detector may be, for example, a detector described in Non Patent Document 2. By using the semiconductor detector, it is expected that the energy resolution is improved, and as a result, the removal capability of scattering components is improved, and the image quality is improved.

When the fact that the spatial resolution of the tomographic image acquired by the nuclear medicine diagnostic apparatus such as the PET apparatus is about 3 to 5 mm is considered, it is desirable that the spatial resolution required for each of the first detector 11 and the second detector 12 is equal to the above, or better than the above. Similarly, it is desirable that the temporal resolution required for each of the first detector 11 and the second detector 12 is 20 to 35 ps or less in the coincidence detection temporal resolution.

In the case in which the Cherenkov radiator or the scintillator is included, it is preferable that each of the first detector 11 and the second detector 12 outputs a signal indicating a position (detection position) and a time (detection time) at which the gamma-ray interacts in the Cherenkov radiator or the scintillator, instead of a position and a time at which the Cherenkov light or the scintillation light is detected. In this case, the detection position is indicated by three-dimensional coordinate values for specifying not only positions in two directions parallel to a detection surface of the detector but also a position in a direction perpendicular to the detection surface.

It is preferable that the detection surface of each of the first detector 11 and the second detector 12 has a size larger than that of the subject 90 (or a region of interest in the subject 90). For example, in the case of the image acquisition apparatus for acquiring the tomographic image of a human brain, it is preferable that the detection surface of each of the first detector 11 and the second detector 12 has the size similar to or larger than the size of the human brain.

The image acquisition apparatus 1A and an image acquisition method using the apparatus acquire a tomographic image (first tomographic image) of the subject 90 by using a first measurement mode. Further, the image acquisition apparatus 1A and the image acquisition method can acquire a tomographic image (second tomographic image) of the subject 90 by using a second measurement mode.

The first tomographic image is an image representing a distribution of Compton scattering positions in the subject 90, and represents anatomical information of the subject 90. The second tomographic image represents a distribution of positron emitting radionuclides (distribution of a drug) in the subject 90, and can be used for diagnosis of a health condition of the subject 90.

The acquisition of the first tomographic image by the first measurement mode is performed by a first measurement step and a first processing step as described below. FIG. 1 is a diagram illustrating the configuration of the image acquisition apparatus 1A according to the first embodiment, and is in particular a diagram for describing the acquisition of the first tomographic image by using the first measurement mode.

In the first measurement step, the subject 90 is placed between the first detector 11 and the second detector 12. In this case, a positron emitting radionuclide may not be injected into the subject 90. A positron emitting radionuclide 81 is placed between the first detector 11 or the second detector 12 and the subject 90. It is preferable to use the positron emitting radionuclide 81 as small as possible. In this diagram, the positron emitting radionuclide 81 is placed between the first detector 11 and the subject 90.

A positron emitted from the positron emitting radionuclide 81 immediately annihilates with a nearby electron, and a pair of gamma-ray photons traveling in opposite directions are generated by an electron positron annihilation event. When the gamma-ray is detected, each of the first detector 11 and the second detector 12 outputs the signal indicating the detection position and the detection time of the gamma-ray photon.

In the first processing step, the processing unit 20, for each coincidence event in which the first detector 11 and the second detector 12 perform coincidence detection of the pair of gamma-ray photons which are generated by the electron positron annihilation event in the positron emitting radionuclide 81, assuming that the gamma-ray photon arriving at one detector out of the first detector 11 and the second detector 12 is a gamma-ray photon arriving without being Compton scattered in the subject, and the gamma-ray photon arriving at the other detector is a gamma-ray photon arriving after being Compton scattered in the subject, determines a position at which the gamma-ray photon is Compton scattered based on the detection position and the detection time of the gamma-ray photon by each of the first detector 11 and the second detector 12 and the position of the positron emitting radionuclide 81.

Further, the processing unit 20 creates the first tomographic image representing the distribution of the Compton scattering positions in the subject 90 which are respectively determined for the plurality of coincidence events. The above first tomographic image is an image representing the anatomical information of the subject 90.

The processing unit 20 can determine whether or not the gamma-ray photon arriving at the first detector 11 or the second detector 12 is a gamma-ray photon arriving after the Compton scattering, based on any one or more of the position of the positron emitting radionuclide 81, the magnitude of the energy of the gamma-ray photon, and the detection time of the gamma-ray photon by each of the first detector 11 and the second detector 12.

As illustrated in FIG. 1, in the case in which the positron emitting radionuclide 81 is placed between the first detector 11 and the subject 90, it is possible to determine that the gamma-ray arriving at the first detector 11 is a gamma-ray without being Compton scattered in the subject. The energy of each of the pair of gamma-ray photons generated by the electron positron annihilation is 511 keV, and further, the energy of the gamma-ray is lowered by the Compton scattering, and thus, it is possible to determine whether or not the gamma-ray arrives after the Compton scattering based on the magnitude of the energy of the gamma-ray. It is possible to determine whether or not the gamma-ray photon arrives after the Compton scattering in the subject based on the temporal relationship between the detection times of the gamma-ray photons respectively by the first detector 11 and the second detector 12.

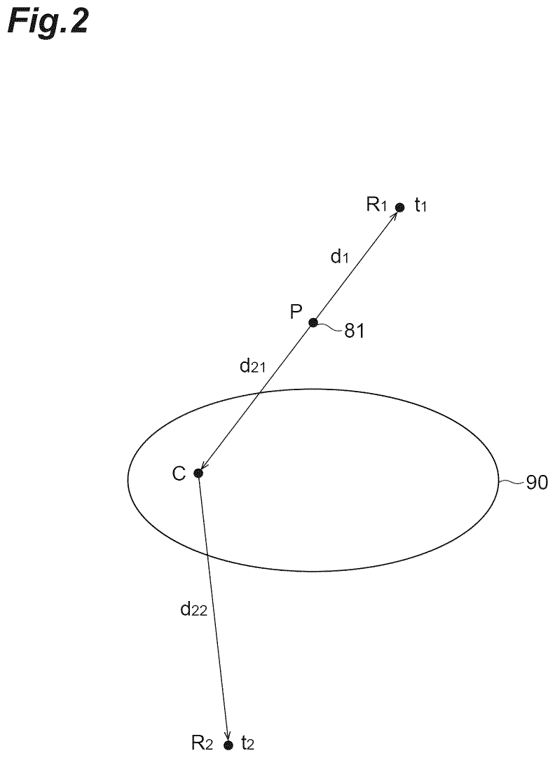

FIG. 2 is a diagram illustrating a method of determining the position at which the gamma-ray photon is Compton scattered in the subject. The position of the positron emitting radionuclide 81 is set to P, the detection position of the gamma-ray by the first detector 11 is set to R₁, the detection position of the gamma-ray by the second detector 12 is set to R₂, and the position at which the gamma-ray is Compton scattered is set to C. The detection time of the gamma-ray by the first detector 11 is set to t₁, and the detection time of the gamma-ray by the second detector 12 is set to t₂.

Out of the pair of gamma-ray photons which are generated by the electron positron annihilation event in the positron emitting radionuclide 81, a flight distance of one gamma-ray is a distance d₁ from the position P to the position R₁. A flight distance of the other gamma-ray is a sum (d₂₁+d₂₂) of a distance d₂₁ from the position P to the position C and a distance d₂₂ from the position C to the position R₂.

A difference (d₂₁+d₂₂−d₁) of the flight distances of the pair of gamma-ray photons is equal to a value obtained by multiplying a difference (t₂−t₁) of the detection times of the gamma-ray photons by the light speed c. Further, a line segment connecting the position P and the position R₁ and a line segment connecting the position P and the position C are parallel to each other. As described above, the position C at which the gamma-ray photon is Compton scattered can be determined based on the detection position R₁ and the detection time t₁ of the gamma-ray photon by the first detector 11, the detection position R₂ and the detection time t₂ of the gamma-ray photon by the second detector 12, and the position P of the positron emitting radionuclide 81.

In the case in which both the pair of gamma-ray photons are gamma-ray photons without being Compton scattered, the Compton scattering position determined by the method described with reference to FIG. 2 coincides with the position P of the positron emitting radionuclide 81. The above position is a position outside the subject 90, and thus, it can be easily excluded.

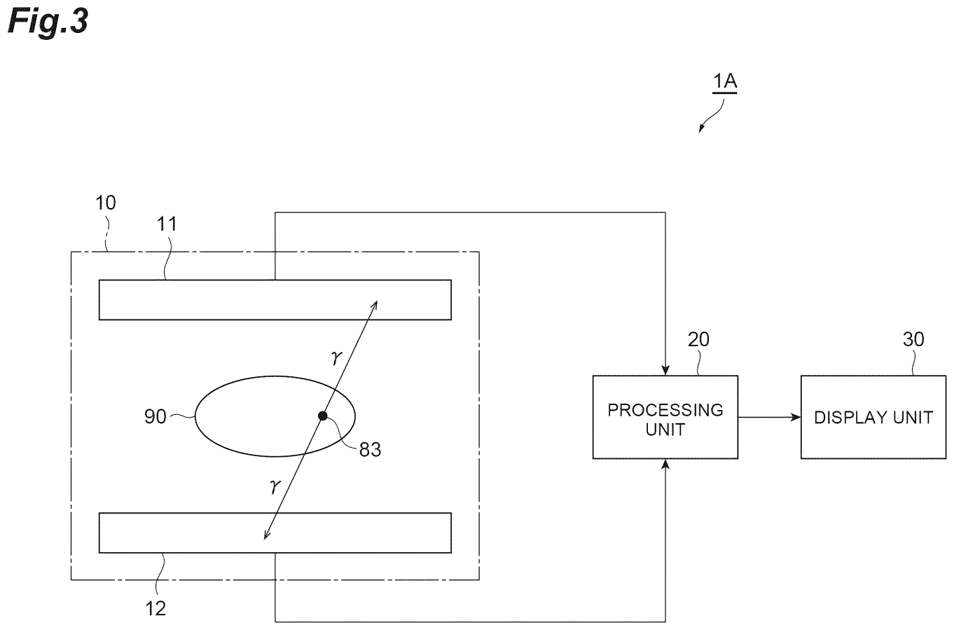

The acquisition of the second tomographic image by the second measurement mode is performed by a second measurement step and a second processing step as described below. FIG. 3 is a diagram illustrating the configuration of the image acquisition apparatus 1A of the first embodiment, and is in particular a diagram for describing the acquisition of the second tomographic image by using the second measurement mode.

In the second measurement step, the subject 90 into which a drug labeled with a positron emitting radionuclide 83 is injected is placed between the first detector 11 and the second detector 12. In this case, a positron emitting radionuclide may not be placed outside the subject 90. A pair of gamma-ray photons traveling in opposite directions are generated by the electron positron annihilation event in the positron emitting radionuclide 83 labeling the drug which is injected into the subject 90. When the gamma-ray is detected, each of the first detector 11 and the second detector 12 outputs the signal indicating the detection position and the detection time of the gamma-ray photon.

It is preferable that the positron emitting radionuclide 83 labeling the drug which is injected into the subject 90 in the second measurement step is a positron emitting radionuclide of the same type as the positron emitting radionuclide 81 placed between the first detector 11 and the subject 90 in the first measurement step. By using the positron emitting radionuclides of the same type as the positron emitting radionuclide 83 and the positron emitting radionuclide 81, only one type of the positron emitting radionuclide needs to be prepared, and thus, the measurement preparation becomes easy. In addition, the positron emitting radionuclide 81 may be set to a positron emitting radionuclide for calibration, such as 68Ge/68Ga.

In the second processing step, the processing unit 20, for each coincidence event in which the first detector 11 and the second detector 12 perform coincidence detection of the pair of gamma-ray photons which are generated by the electron positron annihilation event in the positron emitting radionuclide 83, determines a position at which the annihilation event occurs based on the detection position and the detection time of the gamma-ray photon by each of the first detector 11 and the second detector 12. The annihilation event occurrence position for each of the coincidence events can be determined based on a difference of the detection times of the gamma-ray photons respectively detected by the first detector 11 and the second detector 12 on a line segment connecting the detection positions of the gamma-ray photons respectively detected by the first detector 11 and the second detector 12.

Further, the processing unit 20 creates the second tomographic image representing the distribution of the annihilation event occurrence positions in the subject 90 which are respectively determined for the plurality of coincidence events. The temporal resolution of the first detector 11 and the second detector 12 is good, and thus, the second tomographic image, which is a three-dimensional tomographic image, can be acquired without performing the image reconstruction processing.

The above second tomographic image is an image representing the distribution of the positron emitting radionuclides 83 (the distribution of the drug) in the subject 90, and can be used for performing diagnosis of the health condition of the subject 90. In addition, by correcting the second tomographic image based on the first tomographic image, the processing unit 20 can further acquire the second tomographic image after the correction is performed for a gamma-ray absorption distribution in the subject 90.

Any one of the acquisition of the first tomographic image in the first measurement mode and the acquisition of the second tomographic image in the second measurement mode may be performed first. In addition, in the case in which the acquisition of the second tomographic image in the second measurement mode is performed first, the annihilation event in the positron emitting radionuclide 83 which is injected into the subject 90 at that time may affect the acquisition of the first tomographic image in the subsequent first measurement mode, and thus, it is preferable to perform the acquisition of the first tomographic image in the first measurement mode first.

In the present embodiment, it is possible to acquire the tomographic image (the first tomographic image) representing the anatomical information of the subject 90 without performing the image reconstruction processing, and thus, it is possible to avoid reduction of the image quality due to the image reconstruction processing, and it is possible to avoid deterioration of the anatomical information. Further, it is possible to acquire the tomographic image (the second tomographic image) representing the distribution of the positron emitting radionuclides (the distribution of the drug) in the subject without performing the image reconstruction processing.

In the present embodiment, a large-scale rotation mechanism such as a mechanism in the X-ray CT apparatus is not required, and thus, a small and inexpensive apparatus can be provided. Further, as compared with the case of using the X-ray CT apparatus, in the present embodiment, an exposure dose of the subject can be reduced.

The apparatus described in Non Patent Document 1 has the configuration of the PET apparatus in which the large number of detectors are arranged around the measurement space in which the subject is placed, and thus, it is difficult to reduce a size, and further, the LSO scintillator containing lutetium Lu which is a rare material is used, and thus, it is difficult to reduce a cost. On the other hand, in the present embodiment, the above problems do not occur, and it is possible to realize the size reduction and the cost reduction.

The apparatus described in Patent Document 1 uses the gas detector, and thus, it is difficult to improve detection efficiency. On the other hand, in the present embodiment, the above problem does not occur, and it is possible to improve the detection efficiency.

### Second Embodiment

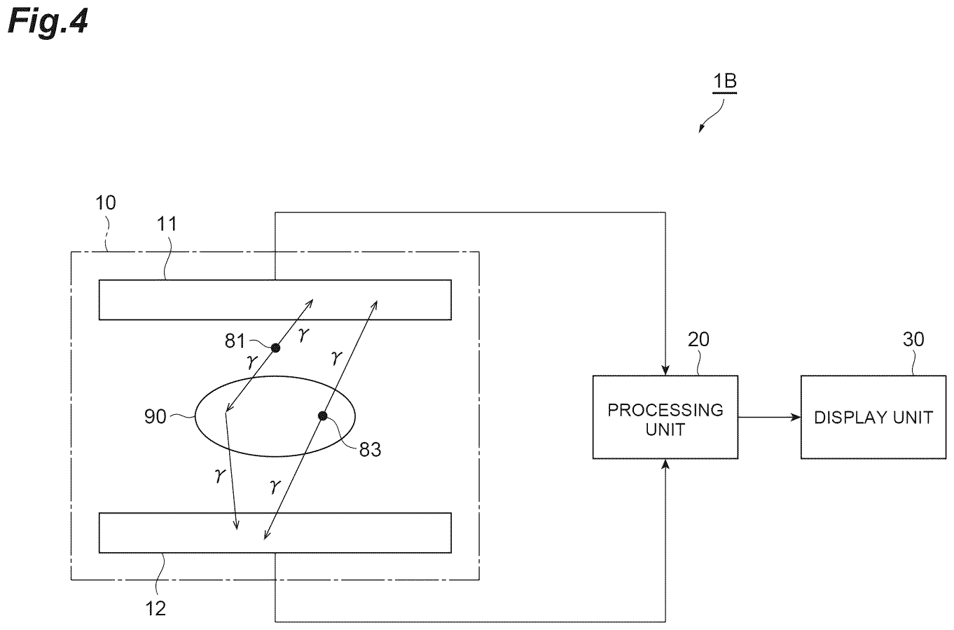

FIG. 4 is a diagram illustrating a configuration of an image acquisition apparatus 1B according to a second embodiment. The image acquisition apparatus 1B includes the measurement unit 10, the processing unit 20, and the display unit 30. As compared with the first embodiment, the second embodiment is different in that the Compton scattering position in the subject 90 and the annihilation event occurrence position in the subject 90 are determined in a common period.

In the measurement step, the subject 90 into which the drug labeled with the positron emitting radionuclide 83 is injected is placed between the first detector 11 and the second detector 12. Further, the positron emitting radionuclide 81 is placed between the first detector 11 or the second detector 12 and the subject 90. In this diagram, the positron emitting radionuclide 81 is placed between the first detector 11 and the subject 90.

By using the positron emitting radionuclides of the same type as the positron emitting radionuclide 83 labeling the drug which is injected into the subject 90 and the positron emitting radionuclide 81 placed between the first detector 11 and the subject 90, only one type of the positron emitting radionuclide needs to be prepared, and thus, the measurement preparation becomes easy. In addition, the positron emitting radionuclide 81 may be set to a positron emitting radionuclide for calibration, such as 68Ge/68Ga.

The pair of gamma-ray photons traveling in opposite directions are generated by the electron positron annihilation event in each of the positron emitting radionuclide 81 and the positron emitting radionuclide 83. When the gamma-ray is detected, each of the first detector 11 and the second detector 12 outputs the signal indicating the detection position and the detection time of the gamma-ray photon.

In the processing step, the processing unit 20, for each coincidence event in which the first detector 11 and the second detector 12 perform coincidence detection of the pair of gamma-ray photons which are generated by the electron positron annihilation event in the positron emitting radionuclide 81 or the positron emitting radionuclide 83, performs the following processing.

The processing unit 20 determines whether or not the gamma-ray photon arriving at the first detector 11 or the second detector 12 is a gamma-ray photon arriving after the Compton scattering, based on any one or more of the position of the positron emitting radionuclide 81, the magnitude of the energy of the gamma-ray photon, and the detection time of the gamma-ray photon by each of the first detector 11 and the second detector 12.

The processing unit 20, as a result of the above determination, in the case in which the gamma-ray photon arriving at one detector out of the first detector 11 and the second detector 12 is a gamma-ray photon arriving without being Compton scattered in the subject, and the gamma-ray photon arriving at the other detector is a gamma-ray photon arriving after being Compton scattered in the subject, assuming that each of the pair of gamma-ray photons arrives from the positron emitting radionuclide 81 outside the subject 90, by using the calculation described with reference to FIG. 2, determines the position at which the gamma-ray photon is Compton scattered in the subject 90 based on the detection position and the detection time of the gamma-ray photon by each of the first detector 11 and the second detector 12 and the position of the positron emitting radionuclide 81.

On the other hand, the processing unit 20, as a result of the above determination, in the case in which the gamma-ray photons arriving at both the first detector 11 and the second detector 12 are gamma-ray photons arriving without being Compton scattered in the subject, assuming that each of the pair of gamma-ray photons arrives from the positron emitting radionuclide 83 in the subject 90, determines the position at which the annihilation event occurs in the subject 90 based on the detection position and the detection time of the gamma-ray photon by each of the first detector 11 and the second detector 12.

Further, after performing the above processing for the plurality of coincidence events, the processing unit 20 creates the first tomographic image representing the distribution of the Compton scattering positions in the subject 90, and further, creates the second tomographic image representing the distribution of the annihilation event occurrence positions in the subject 90.

The first tomographic image is an image representing the anatomical information of the subject 90. The second tomographic image is an image representing the distribution of the positron emitting radionuclides 83 (the distribution of the drug) in the subject 90, and can be used for performing diagnosis of the health condition of the subject 90. In addition, by correcting the second tomographic image based on the first tomographic image, the processing unit 20 can further acquire the second tomographic image after the correction is performed for the gamma-ray absorption distribution in the subject 90.

In the second embodiment, in addition to the same effect as that in the first embodiment, the Compton scattering position in the subject 90 and the annihilation event occurrence position in the subject 90 can be determined in a common period, and thus, the time for constraining the subject 90 can be shortened.

### Third Embodiment

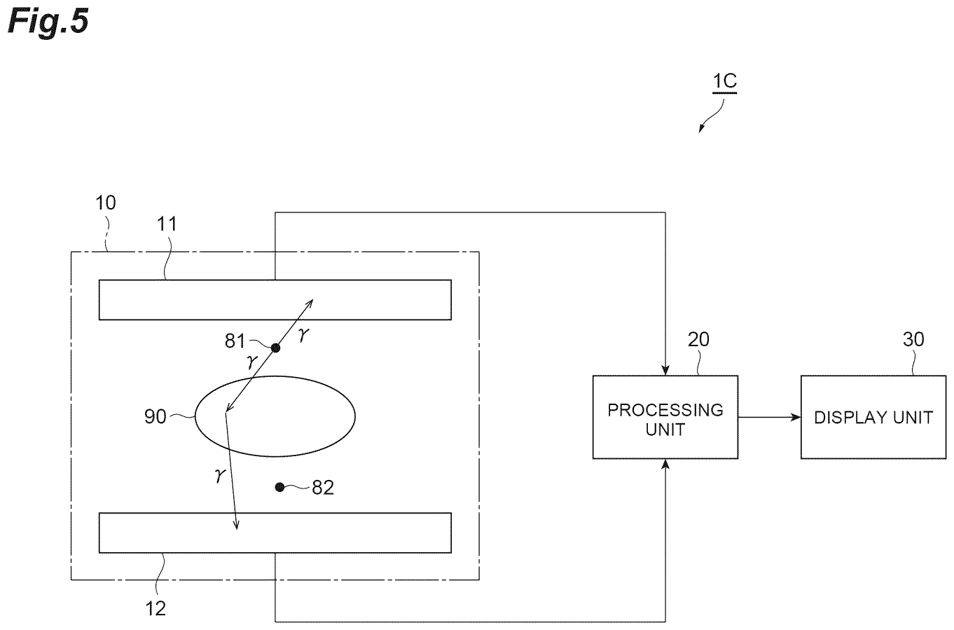

FIG. 5 is a diagram illustrating a configuration of an image acquisition apparatus 1C according to a third embodiment. The image acquisition apparatus 1C includes the measurement unit 10, the processing unit 20, and the display unit 30. As compared with the embodiments described above, the third embodiment is different in that the positron emitting radionuclide 81 is placed between the first detector 11 and the subject 90, and further, a positron emitting radionuclide 82 is also placed between the second detector 12 and the subject 90. It is preferable that the positron emitting radionuclide 81 and the positron emitting radionuclide 82 are the positron emitting radionuclides of the same type.

The processing unit 20 determines whether or not the gamma-ray photon arriving at the first detector 11 or the second detector 12 is a gamma-ray photon arriving after the Compton scattering, and whether the gamma-ray photon is generated from the positron emitting radionuclide 81 or the positron emitting radionuclide 82, based on any one or more of the positions of the positron emitting radionuclides 81 and 82, the magnitude of the energy of the gamma-ray photon, and the detection time of the gamma-ray photon by each of the first detector 11 and the second detector 12. The processing unit 20, based on the determination result, determines the position at which the gamma-ray photon is Compton scattered in the subject 90.

In the present embodiment, the positron emitting radionuclides 81 and 82 can be arranged with good symmetry with respect to the subject 90, and thus, the first tomographic image with better quality can be acquired. Further, the number of Compton scattering events per unit time in the subject 90 increases, and thus, the measurement time can be shortened.

### Fourth Embodiment

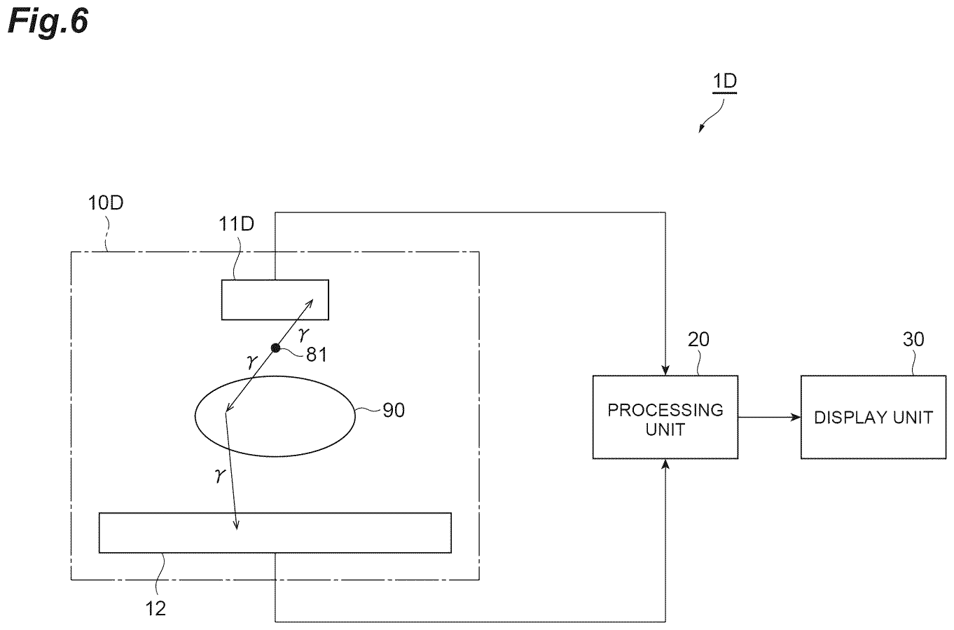

FIG. 6 is a diagram illustrating a configuration of an image acquisition apparatus 1D according to a fourth embodiment. The image acquisition apparatus 1D includes a measurement unit 10D, the processing unit 20, and the display unit 30. As compared with the embodiments described above, the image acquisition apparatus 1D of the fourth embodiment is different in that the measurement unit 10D is provided instead of the measurement unit 10.

The measurement unit 10D includes a first detector 11D and the second detector 12. A detection surface of the first detector 11D is narrower than a detection surface of the second detector 12. The positron emitting radionuclide 81 is placed between the first detector 11D and the subject 90. Each of the first detector 11D and the second detector 12 outputs the signal indicating the detection position and the detection time when the gamma-ray photon is detected.

In order to acquire the first tomographic image, it is only required that one gamma-ray photon out of the pair of gamma-ray photons generated by the electron positron annihilation event in the positron emitting radionuclide 81 is incident on the first detector 11D, and the other gamma-ray photon is incident on the subject 90 (or the region of interest in the subject 90), and thus, the detection surface of the first detector 11D can be narrowed as long as the above condition is satisfied. As the position at which the positron emitting radionuclide 81 is placed is closer to the first detector 11D, the detection surface of the first detector 11D can be made narrower. As described above, the size of the first detector 11D can be reduced, and thus, the image acquisition apparatus 1D can be configured at low cost.

### Fifth Embodiment

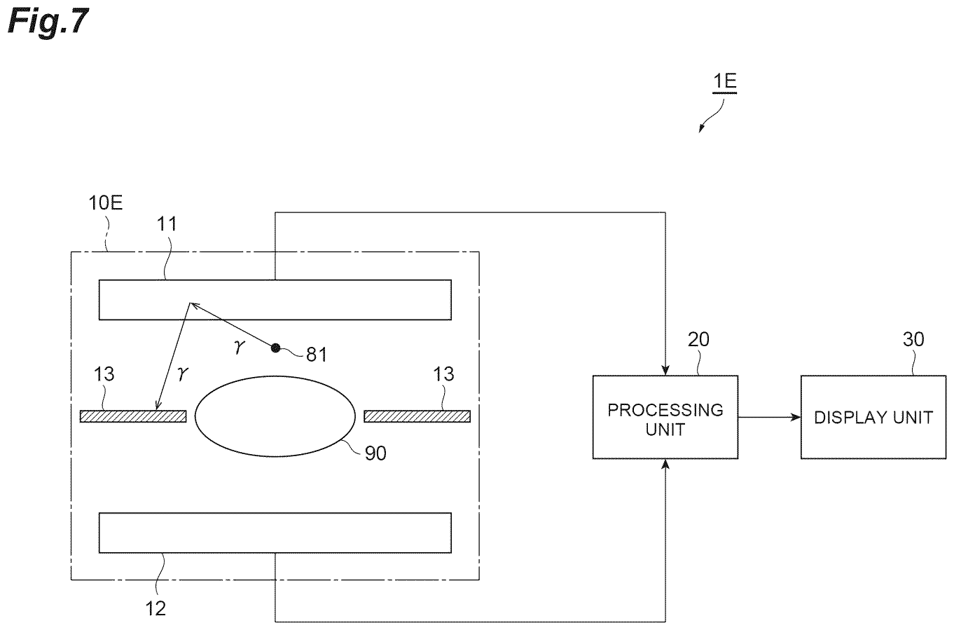

FIG. 7 is a diagram illustrating a configuration of an image acquisition apparatus 1E according to a fifth embodiment. The image acquisition apparatus 1E includes a measurement unit 10E, the processing unit 20, and the display unit 30. As compared with the embodiments described above, the image acquisition apparatus 1E of the fifth embodiment is different in that the measurement unit 10E is provided instead of the measurement unit 10.

The measurement unit 10E includes a shield 13 in addition to the first detector 11 and the second detector 12. The shield 13 is provided for preventing a gamma-ray photon backscattered in one detector out of the first detector 11 and the second detector 12 from being incident on the other detector.

The shield 13 is arranged between the first detector 11 and the second detector 12 and at a position at which the measurement of the Compton scattering position in the subject 90 and the measurement of the annihilation event occurrence position in the subject 90 are not blocked. It is preferable that the shield 13 is a plate-shaped member made of a high density material (for example, lead) capable of blocking the gamma-ray.

### Sixth Embodiment

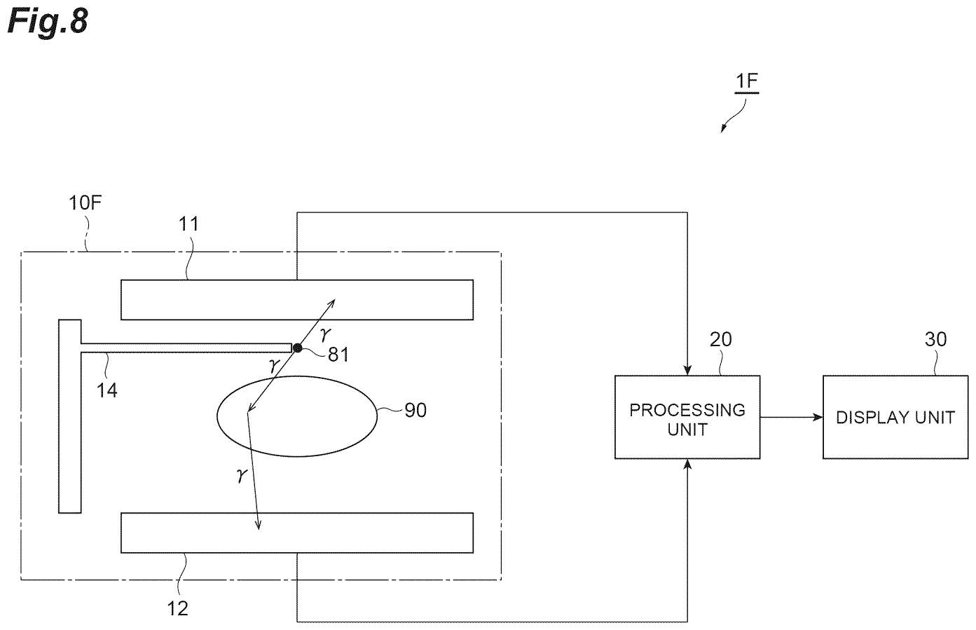

FIG. 8 is a diagram illustrating a configuration of an image acquisition apparatus 1F according to a sixth embodiment. The image acquisition apparatus 1F includes a measurement unit 10F, the processing unit 20, and the display unit 30. As compared with the embodiments described above, the image acquisition apparatus 1F of the sixth embodiment is different in that the measurement unit 10F is provided instead of the measurement unit 10.

The measurement unit 10F includes a moving unit 14 in addition to the first detector 11 and the second detector 12. The moving unit 14 is a unit for moving the positron emitting radionuclide 81 in a space between the first detector 11 or the second detector 12 and the subject 90. The moving unit 14 may move the positron emitting radionuclide 81 continuously with the lapse of time, or may move the positron emitting radionuclide 81 so as to arrange the positron emitting radionuclide 81 sequentially at separate positions. The processing unit 20 constantly identifies the position of the positron emitting radionuclide 81 in order to determine the position at which the gamma-ray photon is Compton scattered in the subject 90.

A moving direction of the positron emitting radionuclide 81 may be one direction or two directions parallel to the detection surface of the first detector 11 or the second detector 12, may be a direction perpendicular to the detection surface, or may be three directions including directions parallel to the detection surface and a direction perpendicular to the detection surface. By moving the positron emitting radionuclide 81 in the direction parallel to the detection surface, a field of view of the apparatus can be enlarged or uniformized. By moving the positron emitting radionuclide 81 in the direction perpendicular to the detection surface, it is possible to improve the image quality of the first tomographic image to be acquired.

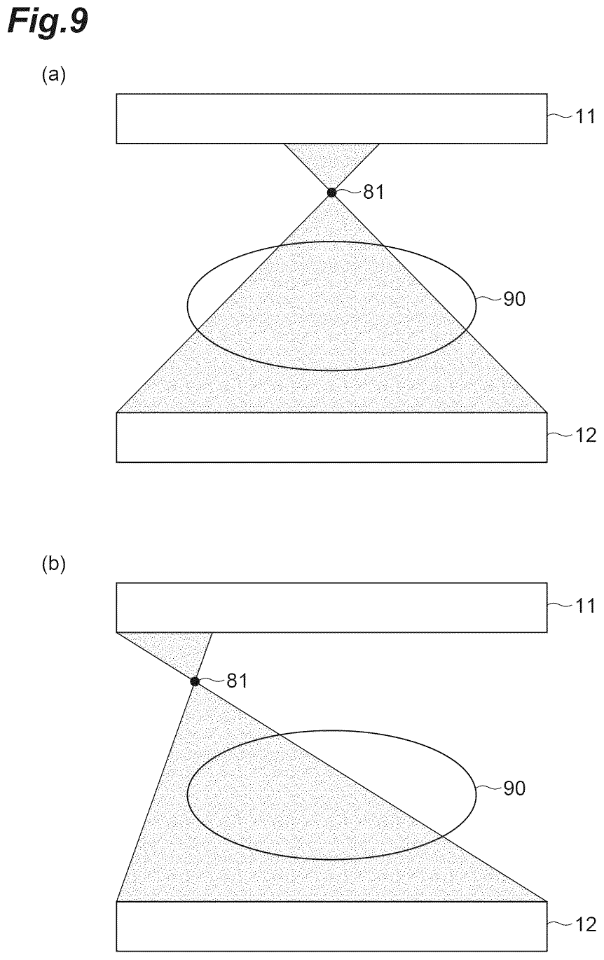

FIG. 9 includes diagrams for describing a change of the field of view of the apparatus in the case in which the positron emitting radionuclide 81 is moved in the direction parallel to the detection surface of the first detector 11 in the measurement unit 10F of the image acquisition apparatus 1F according to the sixth embodiment. In this diagram, the field of view of the apparatus is illustrated by the hatched region. As illustrated in (a) in FIG. 9, in the case in which the positron emitting radionuclide 81 is placed near a center of the detection surface of the first detector 11, both end portions of the subject 90 may fall outside the field of view.

On the other hand, as illustrated in (b) in FIG. 9, in the case in which the positron emitting radionuclide 81 is placed on a first end side of the detection surface of the first detector 11 (the left side with respect to the center in the diagram), the first end side of the subject 90 is included in the field of view, and further, a second end side of the subject 90 (the right side with respect to the center in the diagram) may fall significantly outside the field of view. On the other hand, in the case in which the positron emitting radionuclide 81 is placed on the second end side of the detection surface of the first detector 11, the second end side of the subject 90 is included in the field of view, and further, the first end side of the subject 90 may fall significantly outside the field of view. As described above, by moving the positron emitting radionuclide 81 in the direction parallel to the detection surface of the first detector 11, the field of view of the apparatus can be enlarged or uniformized.

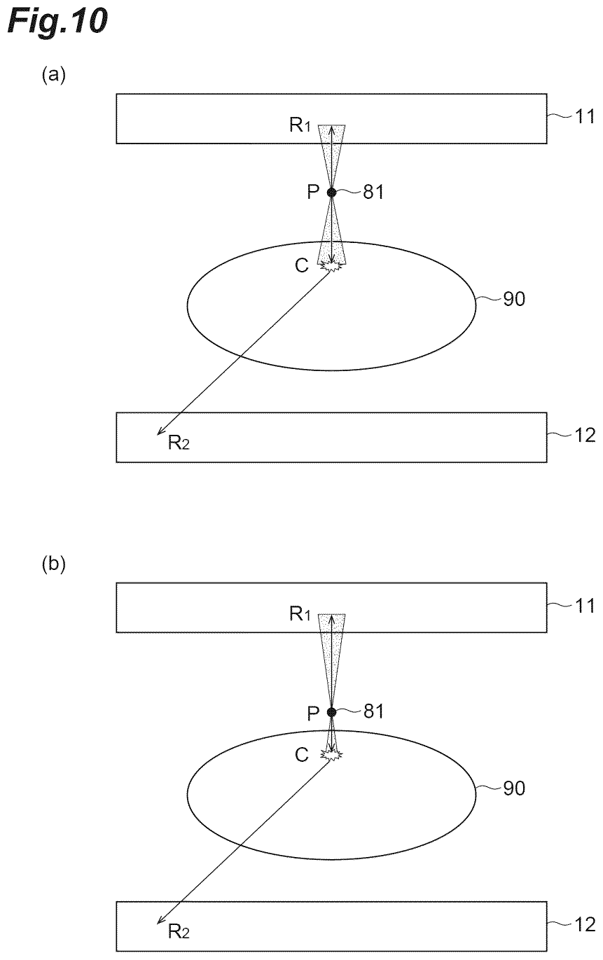

FIG. 10 includes diagrams for describing the image quality of the first tomographic image in the case in which the positron emitting radionuclide 81 is moved in the direction perpendicular to the detection surface of the first detector 11 in the measurement unit 10F of the image acquisition apparatus 1F according to the sixth embodiment. In this diagram, the position of the positron emitting radionuclide 81 is set to P, the detection position of the gamma-ray by the first detector 11 is set to R₁, the detection position of the gamma-ray by the second detector 12 is set to R₂, and the position at which the gamma-ray is Compton scattered is set to C. An error range of the line segment connecting the position P and the position R₁ and an error range of the line segment connecting the position P and the position C are illustrated by the hatched region.

There is an error due to the spatial resolution in the actual detection position R₁ of the gamma-ray by the first detector 11, and thus, an error occurs in the estimation of the line segment connecting the position P and the position R₁, and further, an error occurs also in the estimation of the line segment connecting the position P and the position C, and finally, an error occurs in the estimation of the position C.

As illustrated in (a) in FIG. 10, the longer the distance between the position P and the position C, the larger the estimation error of the position C, and further, as illustrated in (b) in FIG. 10, the shorter the distance between the position P and the position C, the smaller the estimation error of the position C. Therefore, in order to improve the image quality of the first tomographic image to be acquired, it is preferable to place the positron emitting radionuclide 81 at a position close to the subject 90.

The subject 90 has various sizes and shapes, and thus, it is preferable to move the positron emitting radionuclide 81 in the three directions so as to enlarge or uniformize the field of view of the apparatus, and further, improve the image quality of the acquired first tomographic image, according to the size and the shape of the subject 90.

### (Simulation Example)

Next, conditions and results of a simulation performed for the acquisition of the first tomographic image of the subject (the image representing the distribution of the Compton scattering positions in the subject) described with reference to FIG. 1 and FIG. 2 will be described. In this case, Geant4 capable of simulating tracks of particles in a material by using a Monte Carlo method is used.

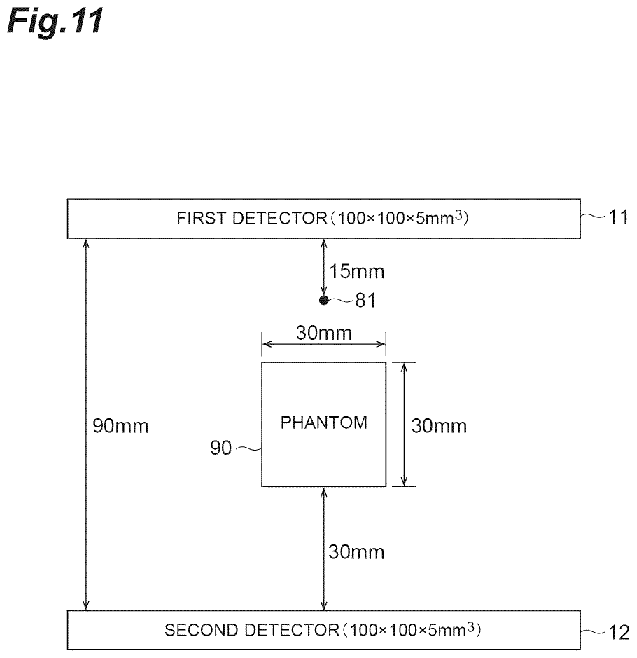

FIG. 11 is a diagram illustrating a configuration and an arrangement of the measurement unit which is assumed in the simulation. Each of the first detector 11 and the second detector 12 is set to a Cherenkov detector including a Cherenkov radiator having a size of 100× 100×5 mm³. Both the temporal resolution and the spatial resolution of the gamma-ray detection by each of the first detector 11 and the second detector 12 are respectively set to 0 which is an ideal value. The first detector 11 and the second detector 12 are arranged facing each other in parallel with a distance of 90 mm therebetween.

A phantom having a cylindrical shape with a diameter of 30 mm and a height of 30 mm is assumed, and the phantom is set as the subject 90. The subject 90 is placed at the center position between the first detector 11 and the second detector 12 such that a center axis of the cylindrical shape of the subject 90 is perpendicular to the detection surface of each of the first detector 11 and the second detector 12. The positron emitting radionuclide 81 is placed at the center position between the first detector 11 and the subject 90. A size of the positron emitting radionuclide 81 is ignored.

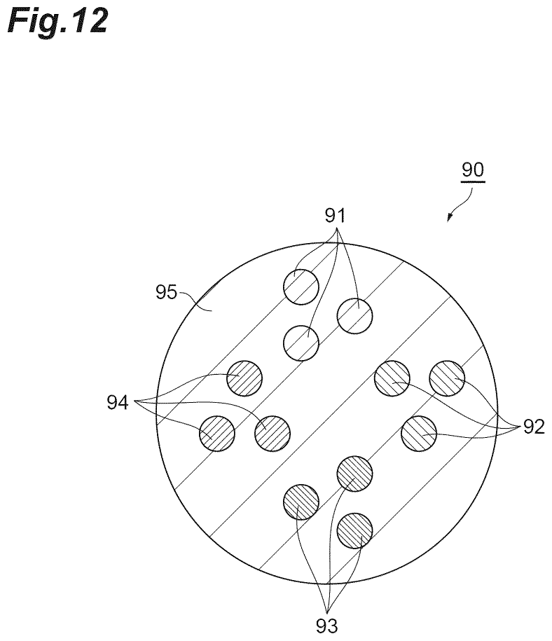

FIG. 12 is a diagram illustrating a configuration of the phantom assumed as the subject 90 in the simulation. This diagram is a view of the phantom as the subject 90 having the cylindrical shape viewed in the center axis direction. In the subject 90, a region 91, a region 92, a region 93, and a region 94, each having a cylindrical shape with a diameter of 3 mm, extend in a direction parallel to the center axis of the cylindrical shape, and these regions are covered with a region 95 having a cylindrical shape with a diameter of 30 mm.

Each of the region 91, the region 92, the region 93, and the region 94 is provided with three regions. The region 91 is set to a region made of iodine (atomic number 53). The region 92 is set to a region made of air. The region 93 is set to a region made of gadolinium (atomic number 64). The region 94 is set to a region made of BGO as an example of a heavy material. The region 95 is set to a region made of water.

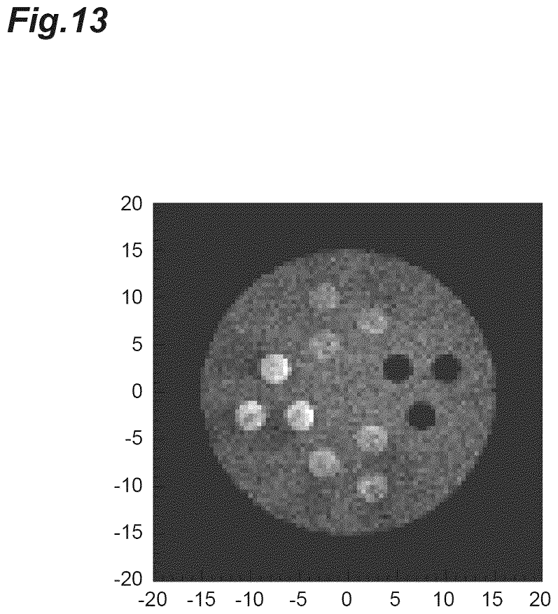

FIG. 13 is a diagram showing the first tomographic image which is acquired in the simulation. This diagram is an image of a cross-section perpendicular to the center axis of the phantom as the subject 90 having the cylindrical shape. In this diagram, the Compton scattering occurrence frequency is represented by grayscale, and the larger the Compton scattering occurrence frequency, the lighter the color. As shown in this diagram, the first tomographic image acquired in the simulation shows that the Compton scattering occurrence frequency is higher in the order of the region 94 (BGO), the region 93 (gadolinium), the region 91 (iodine), the region 95 (water), and the region 92 (air).

As described above, it is confirmed that the first tomographic image (FIG. 13) representing the distribution of the Compton scattering positions in the subject can be acquired. The Compton scattering occurrence probability in the material is proportional to the atomic number of the material. Therefore, it can be said that the first tomographic image represents the distribution of the absorption coefficient ulCompton of the Compton scattering.

The image acquisition apparatus and the image acquisition method are not limited to the embodiments and configuration examples described above, and various modifications are possible. For example, the configurations of any two or more embodiments included in the above embodiments may be combined.

The image acquisition apparatus of a first aspect according to the above embodiment includes (1) a measurement unit including a first detector and a second detector each for detecting a gamma-ray photon, and for outputting a signal indicating a detection position and a detection time when each of the first detector and the second detector detects the gamma-ray photon; and (2) a processing unit for processing the signal output from each of the first detector and the second detector, and (3) the measurement unit, in a first measurement mode, in a state in which a subject is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing unit, in the first measurement mode, for each coincidence event in which the first detector and the second detector perform coincidence detection of a pair of gamma-ray photons generated by an electron positron annihilation event in the positron emitting radionuclide, assuming that the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide, and creates a first tomographic image representing a distribution of Compton scattering positions in the subject respectively determined for a plurality of coincidence events.

In the image acquisition apparatus of a second aspect, in the configuration of the first aspect, the measurement unit, in a second measurement mode, in a state in which the subject into which a drug labeled with a positron emitting radionuclide is injected is placed between the first detector and the second detector, outputs the signal indicating the detection position and the detection time of the gamma-ray photon by each of the first detector and the second detector, and the processing unit, in the second measurement mode, for each coincidence event in which the first detector and the second detector perform coincidence detection of a pair of gamma-ray photons generated by an electron positron annihilation event in the positron emitting radionuclide, determines a position at which the annihilation event occurs based on the detection position and the detection time of the gamma-ray photon by each of the first detector and the second detector, creates a second tomographic image representing a distribution of annihilation event occurrence positions in the subject respectively determined for a plurality of coincidence events, and corrects the second tomographic image based on the first tomographic image.

The image acquisition apparatus of a third aspect according to the above embodiment includes (1) a measurement unit including a first detector and a second detector each for detecting a gamma-ray photon, and for outputting a signal indicating a detection position and a detection time when each of the first detector and the second detector detects the gamma-ray photon; and (2) a processing unit for processing the signal output from each of the first detector and the second detector, and (3) the measurement unit, in a state in which a subject into which a drug labeled with a positron emitting radionuclide is injected is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing unit, for each coincidence event in which the first detector and the second detector perform coincidence detection of a pair of gamma-ray photons generated by an electron positron annihilation event in the positron emitting radionuclide, (a) in a case in which the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide placed between the first detector or the second detector and the subject, (b) in a case in which the gamma-ray photons arriving at both the first detector and the second detector are gamma-ray photons arriving without being Compton scattered in the subject, determines a position at which the annihilation event occurs based on the detection position and the detection time of the gamma-ray photon by each of the first detector and the second detector, (c) creates a first tomographic image representing a distribution of Compton scattering positions in the subject respectively determined for a plurality of coincidence events, creates a second tomographic image representing a distribution of annihilation event occurrence positions in the subject respectively determined for a plurality of coincidence events, and corrects the second tomographic image based on the first tomographic image.

In the image acquisition apparatus of a fourth aspect, in the configuration of the second or third aspect, the positron emitting radionuclide placed between the first detector or the second detector and the subject and the positron emitting radionuclide labeling the drug injected into the subject may be the positron emitting radionuclides of the same type.

In the image acquisition apparatus of a fifth aspect, in the configuration of any one of the first to fourth aspects, the processing unit may determine whether or not the gamma-ray photon arriving at the first detector or the second detector is a gamma-ray photon after being Compton scattered, based on any one or more of the position of the positron emitting radionuclide, a magnitude of energy of the gamma-ray photon, and the detection time of the gamma-ray photon by each of the first detector and the second detector.

In the image acquisition apparatus of a sixth aspect, in the configuration of any one of the first to fifth aspects, the measurement unit, in a state in which the positron emitting radionuclide is placed between the first detector and the subject, and a positron emitting radionuclide is placed also between the second detector and the subject, may output the signal indicating the detection position and the detection time of the gamma-ray photon by each of the first detector and the second detector.

In the image acquisition apparatus of a seventh aspect, in the configuration of any one of the first to sixth aspects, the measurement unit, in a state in which a detection surface of the first detector is narrower than a detection surface of the second detector, and the positron emitting radionuclide is placed between the first detector and the subject, may output the signal indicating the detection position and the detection time of the gamma-ray photon by each of the first detector and the second detector.

In the image acquisition apparatus of an eighth aspect, in the configuration of any one of the first to seventh aspects, the measurement unit may further include a shield for preventing a gamma-ray photon backscattered in one detector out of the first detector and the second detector from being incident on another detector.

In the image acquisition apparatus of a ninth aspect, in the configuration of any one of the first to eighth aspects, the measurement unit may further include a moving unit for moving the positron emitting radionuclide in a space between the first detector or the second detector and the subject.

The image acquisition method of a first aspect according to the above embodiment includes (1) a measurement step of using a first detector and a second detector each for detecting a gamma-ray photon, and outputting a signal indicating a detection position and a detection time when each of the first detector and the second detector detects the gamma-ray photon; and (2) a processing step of processing the signal output from each of the first detector and the second detector, and (3) the measurement step, in a first measurement mode, in a state in which a subject is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing step, in the first measurement mode, for each coincidence event in which the first detector and the second detector perform coincidence detection of a pair of gamma-ray photons generated by an electron positron annihilation event in the positron emitting radionuclide, assuming that the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide, and creates a first tomographic image representing a distribution of Compton scattering positions in the subject respectively determined for a plurality of coincidence events.

In the image acquisition method of a second aspect, in the configuration of the first aspect, the measurement step, in a second measurement mode, in a state in which the subject into which a drug labeled with a positron emitting radionuclide is injected is placed between the first detector and the second detector, outputs the signal indicating the detection position and the detection time of the gamma-ray photon by each of the first detector and the second detector, and the processing step, in the second measurement mode, for each coincidence event in which the first detector and the second detector perform coincidence detection of a pair of gamma-ray photons generated by an electron positron annihilation event in the positron emitting radionuclide, determines a position at which the annihilation event occurs based on the detection position and the detection time of the gamma-ray photon by each of the first detector and the second detector, creates a second tomographic image representing a distribution of annihilation event occurrence positions in the subject respectively determined for a plurality of coincidence events, and corrects the second tomographic image based on the first tomographic image.

The image acquisition method of a third aspect according to the above embodiment includes (1) a measurement step of using a first detector and a second detector each for detecting a gamma-ray photon, and outputting a signal indicating a detection position and a detection time when each of the first detector and the second detector detects the gamma-ray photon; and (2) a processing step of processing the signal output from each of the first detector and the second detector, and (3) the measurement step, in a state in which a subject into which a drug labeled with a positron emitting radionuclide is injected is placed between the first detector and the second detector, and a positron emitting radionuclide is placed between the first detector or the second detector and the subject, outputs the signal indicating the detection position and the detection time of the gamma-ray photon by each of the first detector and the second detector, and (4) the processing step, for each coincidence event in which the first detector and the second detector perform coincidence detection of a pair of gamma-ray photons generated by an electron positron annihilation event in the positron emitting radionuclide, (a) in a case in which the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide placed between the first detector or the second detector and the subject, (b) in a case in which the gamma-ray photons arriving at both the first detector and the second detector are gamma-ray photons arriving without being Compton scattered in the subject, determines a position at which the annihilation event occurs based on the detection position and the detection time of the gamma-ray photon by each of the first detector and the second detector, (c) creates a first tomographic image representing a distribution of Compton scattering positions in the subject respectively determined for a plurality of coincidence events, creates a second tomographic image representing a distribution of annihilation event occurrence positions in the subject respectively determined for a plurality of coincidence events, and corrects the second tomographic image based on the first tomographic image.

In the image acquisition method of a fourth aspect, in the configuration of the second or third aspect, the positron emitting radionuclide placed between the first detector or the second detector and the subject and the positron emitting radionuclide labeling the drug injected into the subject may be the positron emitting radionuclides of the same type.

In the image acquisition method of a fifth aspect, in the configuration of any one of the first to fourth aspects, the processing step may determine whether or not the gamma-ray photon arriving at the first detector or the second detector is a gamma-ray photon after being Compton scattered, based on any one or more of the position of the positron emitting radionuclide, a magnitude of energy of the gamma-ray photon, and the detection time of the gamma-ray photon by each of the first detector and the second detector.

In the image acquisition method of a sixth aspect, in the configuration of any one of the first to fifth aspects, the measurement step, in a state in which the positron emitting radionuclide is placed between the first detector and the subject, and a positron emitting radionuclide is placed also between the second detector and the subject, may output the signal indicating the detection position and the detection time of the gamma-ray photon by each of the first detector and the second detector.

In the image acquisition method of a seventh aspect, in the configuration of any one of the first to sixth aspects, the measurement step, in a state in which the first detector with a detection surface narrower than a detection surface of the second detector is used, and the positron emitting radionuclide is placed between the first detector and the subject, may output the signal indicating the detection position and the detection time of the gamma-ray photon by each of the first detector and the second detector.

In the image acquisition method of an eighth aspect, in the configuration of any one of the first to seventh aspects, the measurement step may prevent a gamma-ray photon backscattered in one detector out of the first detector and the second detector from being incident on another detector by using a shield.

In the image acquisition method of a ninth aspect, in the configuration of any one of the first to eighth aspects, the measurement step may move the positron emitting radionuclide in a space between the first detector or the second detector and the subject.

### INDUSTRIAL APPLICABILITY

The present invention can be used as an image acquisition apparatus and an image acquisition method capable of acquiring a tomographic image representing anatomical information of a subject without performing image reconstruction processing.

### REFERENCE SIGNS LIST

1A-1F—image acquisition apparatus, 10, 10D, 10E, 10F—measurement unit, 11, 11D—first detector, 12—second detector, 13—shield, 14—moving unit, 20—processing unit, 30—display unit, 81, 82, 83—positron emitting radionuclide, 90-subject.

1. An image acquisition apparatus comprising:
a measurement unit including a first detector and a second detector each configured to detect a gamma-ray photon, and configured to output a signal indicating a detection position and a detection time when each of the first detector and the second detector detects the gamma-ray photon; and
a processing unit configured to process the signal output from each of the first detector and the second detector, wherein
the measurement unit, in a first measurement mode, in a state in which a subject is placed between the first detector and the second detector, and a positron emitting radionuclide is placed between the first detector or the second detector and the subject, outputs the signal indicating the detection position and the detection time of the gamma-ray photon by each of the first detector and the second detector, and
the processing unit, in the first measurement mode,
for each coincidence event in which the first detector and the second detector perform coincidence detection of a pair of gamma-ray photons generated by an electron positron annihilation event in the positron emitting radionuclide,
assuming that the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide, and creates a first tomographic image representing a distribution of Compton scattering positions in the subject respectively determined for a plurality of coincidence events.

2. The image acquisition apparatus according to claim 1, wherein
the measurement unit, in a second measurement mode, in a state in which the subject into which a drug labeled with a positron emitting radionuclide is injected is placed between the first detector and the second detector, outputs the signal indicating the detection position and the detection time of the gamma-ray photon by each of the first detector and the second detector, and
the processing unit, in the second measurement mode,
for each coincidence event in which the first detector and the second detector perform coincidence detection of a pair of gamma-ray photons generated by an electron positron annihilation event in the positron emitting radionuclide,
determines a position at which the annihilation event occurs based on the detection position and the detection time of the gamma-ray photon by each of the first detector and the second detector, creates a second tomographic image representing a distribution of annihilation event occurrence positions in the subject respectively determined for a plurality of coincidence events, and corrects the second tomographic image based on the first tomographic image.

3. An image acquisition apparatus comprising:
a measurement unit including a first detector and a second detector each configured to detect a gamma-ray photon, and configured to output a signal indicating a detection position and a detection time when each of the first detector and the second detector detects the gamma-ray photon; and
a processing unit configured to process the signal output from each of the first detector and the second detector, wherein
the measurement unit, in a state in which a subject into which a drug labeled with a positron emitting radionuclide is injected is placed between the first detector and the second detector, and a positron emitting radionuclide is placed between the first detector or the second detector and the subject, outputs the signal indicating the detection position and the detection time of the gamma-ray photon by each of the first detector and the second detector, and
the processing unit,
for each coincidence event in which the first detector and the second detector perform coincidence detection of a pair of gamma-ray photons generated by an electron positron annihilation event in the positron emitting radionuclide,
in a case in which the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide placed between the first detector or the second detector and the subject,
in a case in which the gamma-ray photons arriving at both the first detector and the second detector are gamma-ray photons arriving without being Compton scattered in the subject, determines a position at which the annihilation event occurs based on the detection position and the detection time of the gamma-ray photon by each of the first detector and the second detector,
creates a first tomographic image representing a distribution of Compton scattering positions in the subject respectively determined for a plurality of coincidence events, creates a second tomographic image representing a distribution of annihilation event occurrence positions in the subject respectively determined for a plurality of coincidence events, and corrects the second tomographic image based on the first tomographic image.

4. The image acquisition apparatus according to claim 2, wherein the positron emitting radionuclide placed between the first detector or the second detector and the subject and the positron emitting radionuclide labeling the drug injected into the subject are the positron emitting radionuclides of the same type.

5. The image acquisition apparatus according to claim 1, wherein the processing unit is configured to determine whether or not the gamma-ray photon arriving at the first detector or the second detector is a gamma-ray photon after being Compton scattered, based on any one or more of the position of the positron emitting radionuclide, a magnitude of energy of the gamma-ray photon, and the detection time of the gamma-ray photon by each of the first detector and the second detector.

6. The image acquisition apparatus according to claim 1, wherein the measurement unit, in a state in which the positron emitting radionuclide is placed between the first detector and the subject, and a positron emitting radionuclide is placed also between the second detector and the subject, outputs the signal indicating the detection position and the detection time of the gamma-ray photon by each of the first detector and the second detector.

7. The image acquisition apparatus according to claim 1, wherein the measurement unit, in a state in which a detection surface of the first detector is narrower than a detection surface of the second detector, and the positron emitting radionuclide is placed between the first detector and the subject, outputs the signal indicating the detection position and the detection time of the gamma-ray photon by each of the first detector and the second detector.

8. The image acquisition apparatus according to claim 1, wherein the measurement unit further includes a shield configured to prevent a gamma-ray photon backscattered in one detector out of the first detector and the second detector from being incident on another detector.

9. The image acquisition apparatus according to claim 1, wherein the measurement unit further includes a moving unit configured to move the positron emitting radionuclide in a space between the first detector or the second detector and the subject.

10. An image acquisition method comprising:
a measurement step of using a first detector and a second detector each configured to detect a gamma-ray photon, and outputting a signal indicating a detection position and a detection time when each of the first detector and the second detector detects the gamma-ray photon; and
a processing step of processing the signal output from each of the first detector and the second detector, wherein
the measurement step, in a first measurement mode, in a state in which a subject is placed between the first detector and the second detector, and a positron emitting radionuclide is placed between the first detector or the second detector and the subject, outputs the signal indicating the detection position and the detection time of the gamma-ray photon by each of the first detector and the second detector, and
the processing step, in the first measurement mode,
for each coincidence event in which the first detector and the second detector perform coincidence detection of a pair of gamma-ray photons generated by an electron positron annihilation event in the positron emitting radionuclide,
assuming that the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide, and creates a first tomographic image representing a distribution of Compton scattering positions in the subject respectively determined for a plurality of coincidence events.

11. The image acquisition method according to claim 10, wherein
the measurement step, in a second measurement mode, in a state in which the subject into which a drug labeled with a positron emitting radionuclide is injected is placed between the first detector and the second detector, outputs the signal indicating the detection position and the detection time of the gamma-ray photon by each of the first detector and the second detector, and
the processing step, in the second measurement mode,
for each coincidence event in which the first detector and the second detector perform coincidence detection of a pair of gamma-ray photons generated by an electron positron annihilation event in the positron emitting radionuclide,
determines a position at which the annihilation event occurs based on the detection position and the detection time of the gamma-ray photon by each of the first detector and the second detector, creates a second tomographic image representing a distribution of annihilation event occurrence positions in the subject respectively determined for a plurality of coincidence events, and corrects the second tomographic image based on the first tomographic image.

12. An image acquisition method comprising:
a measurement step of using a first detector and a second detector each configured to detect a gamma-ray photon, and outputting a signal indicating a detection position and a detection time when each of the first detector and the second detector detects the gamma-ray photon; and
a processing step of processing the signal output from each of the first detector and the second detector, wherein
the measurement step, in a state in which a subject into which a drug labeled with a positron emitting radionuclide is injected is placed between the first detector and the second detector, and a positron emitting radionuclide is placed between the first detector or the second detector and the subject, outputs the signal indicating the detection position and the detection time of the gamma-ray photon by each of the first detector and the second detector, and
the processing step,
for each coincidence event in which the first detector and the second detector perform coincidence detection of a pair of gamma-ray photons generated by an electron positron annihilation event in the positron emitting radionuclide,
in a case in which the gamma-ray photon arriving at one detector out of the first detector and the second detector is a gamma-ray photon arriving without being Compton scattered in the subject, and the gamma-ray photon arriving at another detector is a gamma-ray photon arriving after being Compton scattered in the subject, determines a position at which the gamma-ray photon is Compton scattered in the subject based on the detection position and the detection time of the gamma-ray photon by each of the first detector and the second detector and a position of the positron emitting radionuclide placed between the first detector or the second detector and the subject,
in a case in which the gamma-ray photons arriving at both the first detector and the second detector are gamma-ray photons arriving without being Compton scattered in the subject, determines a position at which the annihilation event occurs based on the detection position and the detection time of the gamma-ray photon by each of the first detector and the second detector,
creates a first tomographic image representing a distribution of Compton scattering positions in the subject respectively determined for a plurality of coincidence events, creates a second tomographic image representing a distribution of annihilation event occurrence positions in the subject respectively determined for a plurality of coincidence events, and corrects the second tomographic image based on the first tomographic image.

13. The image acquisition method according to claim 11, wherein the positron emitting radionuclide placed between the first detector or the second detector and the subject and the positron emitting radionuclide labeling the drug injected into the subject are the positron emitting radionuclides of the same type.

14. The image acquisition method according to claim 10, wherein the processing step determines whether or not the gamma-ray photon arriving at the first detector or the second detector is a gamma-ray photon after being Compton scattered, based on any one or more of the position of the positron emitting radionuclide, a magnitude of energy of the gamma-ray photon, and the detection time of the gamma-ray photon by each of the first detector and the second detector.

15. The image acquisition method according to claim 10, wherein the measurement step, in a state in which the positron emitting radionuclide is placed between the first detector and the subject, and a positron emitting radionuclide is placed also between the second detector and the subject, outputs the signal indicating the detection position and the detection time of the gamma-ray photon by each of the first detector and the second detector.

16. The image acquisition method according to claim 10, wherein the measurement step, in a state in which the first detector with a detection surface narrower than a detection surface of the second detector is used, and the positron emitting radionuclide is placed between the first detector and the subject, outputs the signal indicating the detection position and the detection time of the gamma-ray photon by each of the first detector and the second detector.

17. The image acquisition method according to claim 10, wherein the measurement step prevents a gamma-ray photon backscattered in one detector out of the first detector and the second detector from being incident on another detector by using a shield.

18. The image acquisition method according to claim 10, wherein the measurement step moves the positron emitting radionuclide in a space between the first detector or the second detector and the subject.

19. The image acquisition apparatus according to claim 3, wherein the positron emitting radionuclide placed between the first detector or the second detector and the subject and the positron emitting radionuclide labeling the drug injected into the subject are the positron emitting radionuclides of the same type.

20. The image acquisition apparatus according to claim 3, wherein the processing unit is configured to determine whether or not the gamma-ray photon arriving at the first detector or the second detector is a gamma-ray photon after being Compton scattered, based on any one or more of the position of the positron emitting radionuclide, a magnitude of energy of the gamma-ray photon, and the detection time of the gamma-ray photon by each of the first detector and the second detector.

21. The image acquisition apparatus according to claim 3, wherein the measurement unit, in a state in which the positron emitting radionuclide is placed between the first detector and the subject, and a positron emitting radionuclide is placed also between the second detector and the subject, outputs the signal indicating the detection position and the detection time of the gamma-ray photon by each of the first detector and the second detector.

22. The image acquisition apparatus according to claim 3, wherein the measurement unit, in a state in which a detection surface of the first detector is narrower than a detection surface of the second detector, and the positron emitting radionuclide is placed between the first detector and the subject, outputs the signal indicating the detection position and the detection time of the gamma-ray photon by each of the first detector and the second detector.

23. The image acquisition apparatus according to claim 3, wherein the measurement unit further includes a shield configured to prevent a gamma-ray photon backscattered in one detector out of the first detector and the second detector from being incident on another detector.

24. The image acquisition apparatus according to claim 3, wherein the measurement unit further includes a moving unit configured to move the positron emitting radionuclide in a space between the first detector or the second detector and the subject.

25. The image acquisition method according to claim 12, wherein the positron emitting radionuclide placed between the first detector or the second detector and the subject and the positron emitting radionuclide labeling the drug injected into the subject are the positron emitting radionuclides of the same type.

26. The image acquisition method according to claim 12, wherein the processing step determines whether or not the gamma-ray photon arriving at the first detector or the second detector is a gamma-ray photon after being Compton scattered, based on any one or more of the position of the positron emitting radionuclide, a magnitude of energy of the gamma-ray photon, and the detection time of the gamma-ray photon by each of the first detector and the second detector.

27. The image acquisition method according to claim 12, wherein the measurement step, in a state in which the positron emitting radionuclide is placed between the first detector and the subject, and a positron emitting radionuclide is placed also between the second detector and the subject, outputs the signal indicating the detection position and the detection time of the gamma-ray photon by each of the first detector and the second detector.

28. The image acquisition method according to claim 12, wherein the measurement step, in a state in which the first detector with a detection surface narrower than a detection surface of the second detector is used, and the positron emitting radionuclide is placed between the first detector and the subject, outputs the signal indicating the detection position and the detection time of the gamma-ray photon by each of the first detector and the second detector.

29. The image acquisition method according to claim 12, wherein the measurement step prevents a gamma-ray photon backscattered in one detector out of the first detector and the second detector from being incident on another detector by using a shield.

30. The image acquisition method according to claim 12, wherein the measurement step moves the positron emitting radionuclide in a space between the first detector or the second detector and the subject.
